(19) United States
(12) Patent Application Publication
(10) Pub. No.: US 2019/0064157 A1
(43) Pub. Date: Feb. 28, 2019

(54) DETECTION DEVICE, DETECTION METHOD, AND ELECTRODE WITH PROBE

(71) Applicant: KABUSHIKI KAISHA TOSHIBA, Tokyo (JP)

(72) Inventors: Takuya MIYAGAWA, Yokohama Kanagawa (JP); Sadato HONGO, Yokohama Kanagawa (JP); Hiroko MIKI, Kawasaki Kanagawa (JP); Hiroshi HAMASAKI, Hiratsuka Kanagawa (JP); Kentaro KOBAYASHI, Tokyo (JP); Ping WANG, Kawasaki Kanagawa (JP); Naofumi NAKAMURA, Tokyo (JP)

(73) Assignee: KABUSHIKI KAISHA TOSHIBA, Tokyo (JP)

(21) Appl. No.: 15/908,700

(22) Filed: Feb. 28, 2018

(30) Foreign Application Priority Data
Aug. 31, 2017 (JP) 2017-167604

(51) Int. Cl. G01N 33/543

(52) U.S. Cl. CPC G01N 33/5438; G01N 27/02

(57) ABSTRACT

According to one embodiment, a detection device is disclosed. The detection device includes a first region, a first electrode on the first region, a second region, a second electrode on the second region, a partition partitioning the first region and the second region and including a through hole, thereby communicating the first region with the second region. The device further includes a probe which binds specifically to the detection target, and is detachably connected to the first electrode, a detacher to detach the probe from the first electrode, a determination unit to determine whether the first liquid contains a detection target based on a change of electrical condition between the first electrode and the second electrode in a state where the first region and the second region are supplied with the first liquid and the second liquid, respectively.

### CROSS-REFERENCE TO RELATED APPLICATIONS

This application is based upon and claims the benefit of priority from Japanese Patent Application No. 2017-167604, filed Aug. 31, 2017, the entire contents of which are incorporated herein by reference.

### FIELD

Embodiments described herein relate generally to a detection device, a detection method for detecting, and an electrode with a probe, which are used to detect a detection target such as viruses and bacteria.

### BACKGROUND

Pandemic of contagious diseases such as influenza is a great threat to the world of these days. Once a pandemic breaks out, there will be so many of patients and an impact to the world economic will be significant. A prevention plan of pandemic is thus a matter of great urgency.

To prevent pandemic, it is important that a patient with a certain disease is diagnosed in its early stage and that such a patient and their contact are isolated and restricted in order to delay the spread of disease. Furthermore, if a disease is correctly diagnosed in its early stage, the treatment of disease can be started before the condition becomes critical. Thus, deaths by the disease can be reduced. In consideration of the above points, performing correct diagnosis of a disease in its early stage is significantly important.

As a method of detecting a pathogen such as virus or bacterium, immunochromatography is used. In this detection method, diagnosis of a contagious disease can be performed simply and rapidly, and thus, it is widely used. However, the minimum detection sensitivity is low in this detection method, and thus, it is not for diagnosis of a disease in its early stage where viruses are not multiplied in a patient's body.

As another detection method, a method using nanopores is known. In this detection method, the minimum detection sensitivity is high. However, when the concentration of pathogen in a sample liquid is low, a time required to detect the pathogen becomes longer.

### BRIEF DESCRIPTION OF THE DRAWINGS

FIG. 1 is a view schematically depicting a detection device according to a first embodiment.

FIG. 2 is a plan view illustrating an example of a partition of the detection device according to the first embodiment.

FIG. 3 is a view schematically depicting a probe of the detection device according to the first embodiment.

FIG. 4 is a diagram for explaining a detection method employing the detection device according to the first embodiment.

FIG. 5 is a view schematically depicting the probe and a detection target bound to the probe.

FIG. 6 is a view for explaining the detection method employing the detection device according to the first embodiment subsequent to FIG. 4.

FIG. 7 is a view schematically depicting the probe which is bound to the detection target and detached from an lower electrode.

FIG. 8 is a view indicating a current signal that differs depending on a presence or absence of the detection target.

FIG. 9 is a view for explaining a detection method employing a detection device of a comparative example.

FIG. 10 is an exploded perspective view of the detection device according to the first embodiment.

FIG. 11A is a cross-sectional view for explaining a manufacturing method of the detection device according to the first embodiment.

FIG. 11B is a cross-sectional view for explaining the manufacturing method of the detection device according to the first embodiment subsequent to FIG. 11A.

FIG. 11C is a cross-sectional view for explaining the manufacturing method of the detection device according to the first embodiment subsequent to FIG. 11B.

FIG. 11D is a cross-sectional view for explaining the manufacturing method of the detection device according to the first embodiment subsequent to FIG. 11C.

FIG. 12 is a cross-sectional view illustrating a variation of FIG. 11D.

FIG. 13 is a view schematically depicting a detection device according to a first variation of the first embodiment.

FIG. 14 is a view schematically depicting a detection device according to a second variation of the first embodiment.

FIG. 15 is a plan view illustrating a partition and a separation electrode of the detection device of the second variation according to the first embodiment.

FIG. 16 is a view schematically depicting a detection device according to a third variation of the first embodiment.

FIG. 17 is a view schematically depicting a detection device according to a fourth variation of the first embodiment.

FIG. 18 is a view schematically depicting a detection device according to a second embodiment.

FIG. 19 is a view for explaining a detection method employing the detection device according to the second embodiment.

FIG. 20 is a view for explaining the detection method employing the detection device according to the second embodiment subsequent to FIG. 19.

FIG. 21 is a view schematically depicting a detection device according to a third embodiment.

FIG. 22 is a view for explaining a detection method employing the detection device according to the third embodiment.

FIG. 23 is a view for explaining the detection method employing the detection device according to the third embodiment subsequent to FIG. 22.

FIG. 24 is a view schematically depicting a detection device according to a fourth embodiment.

FIG. 25 is a view for explaining a detection method employing the detection device according to the fourth embodiment.

FIG. 26A is a plan view for explaining a manufacturing method of the detection device according to the fourth embodiment.

FIG. 26B is a plan view for explaining a manufacturing method of the detection device according to the fourth embodiment subsequent to FIG. 26A.

FIG. 26C is a plan view for explaining a manufacturing method of the detection device according to the fourth embodiment subsequent to FIG. 26B.

FIG. 26D is a plan view for explaining a manufacturing method of the detection device according to the fourth embodiment subsequent to FIG. 26C.

FIG. 27 is a cross-sectional view illustrating a structure in a process of manufacturing the detection device.

FIG. 28 is a view schematically depicting a detection device according to a first variation of the fourth embodiment.

FIG. 29 is a view schematically depicting a detection device according to a second variation of the fourth embodiment.

FIG. 30 is a cross-sectional view illustrating a structure in a process of manufacturing the detection device according to the second variation of the fourth embodiment.

FIG. 31 is a view schematically depicting a detection device according to a third variation of the fourth embodiment.

FIG. 32 is a cross-sectional view illustrating a structure in a process of manufacturing the detection device according to the third variation of the fourth embodiment.

FIG. 33 is a view schematically depicting a detection device according to a fourth variation of fourth embodiment.

FIG. 34 is a cross-sectional view illustrating a structure in a process of manufacturing the detection device according to the fourth variation of the fourth embodiment.

FIG. 35 is a view schematically depicting another probe of the detection device according to an embodiment.

### DETAILED DESCRIPTION

In general, according to one embodiment, a detection device is disclosed. The detection device includes a first region, a first electrode, a second region, a second electrode, a partition, a probe, a detacher, and a determination unit. The first region is to be supplied with a first liquid possibly containing a detection target. The first electrode is provided to the first region. The second region is to be supplied with a second liquid. The second electrode is provided to the second region. The partition partitions the first region and the second region each other, and includes a through hole to communicate the first region and the second region each other. The probe is detachably connected to the first electrode, and binds specifically to the detection target. The detacher detaches the probe from the first electrode. The determination unit determines whether the first liquid contains the detection target based on a change of electrical condition between the first electrode and the second electrode in a state where the first region and the second region are supplied with the first liquid and the second liquid, respectively.

According to another embodiment, a detection method using a detection device is disclosed. The detection device includes a first region, a first electrode provided to the first region, a second region, a second electrode provided to the second region, a partition partitions the first region and the second region each other, and provided with a through hole to communicate the first region and the second region each other, and a probe detachably connected to the first electrode and binds specifically to a detection target. The detection method includes supplying a first liquid and a second liquid to the first region and the second region, respectively, the first liquid possibly containing the detection target; separating the probe from the first electrode; determining whether the first liquid contains the detection target based on a change of electrical condition between the first electrode and the second electrode.

Embodiments will be described hereinafter with reference to the accompanying drawings. The drawings are schematic and conceptual, and the dimensions, the proportions, etc., of each of the drawings are not necessarily the same as those in reality. Further, in the drawings, the same reference symbols denote the same or corresponding portions, and overlapping explanations thereof will be made as necessary. In addition, as used in the description and the appended claims, what is expressed by a singular form shall include the meaning of “more than one.”

### First Embodiment

FIG. 1 is a view schematically depicting a detection device 1 according to the first embodiment.

The detection device 1 includes a vessel 2, and a partition 3 provided therein. The partition partitions the vessel 2 into a first chamber (first region) 11 and a second chamber (second region) 12.

The partition 3 has insulation properties. A material of the partition 3 includes insulating material, for example, glass, sapphire, ceramic, resin, rubber, silicon oxide, silicon nitride, or aluminum oxide.

A through hole 4 is provided to the partition 3, which communicates the first chamber 11 and the second chamber 12 each other, and is a fine hole used as nanopore or micropore. The through hole 4 has a dimension such that a single detection target passes through the through hole 4. Hereafter, the through hole 4 may be referred to as a fine hole 4.

The detection target is, for example, a pathogen such as a virus or bacterium. In addition, the detection target maybe a component of pathogen, for example, a nucleic acid (DNA, RNA), protein, or cell. In the following description, the detection target is an influenza virus that is one of viruses.

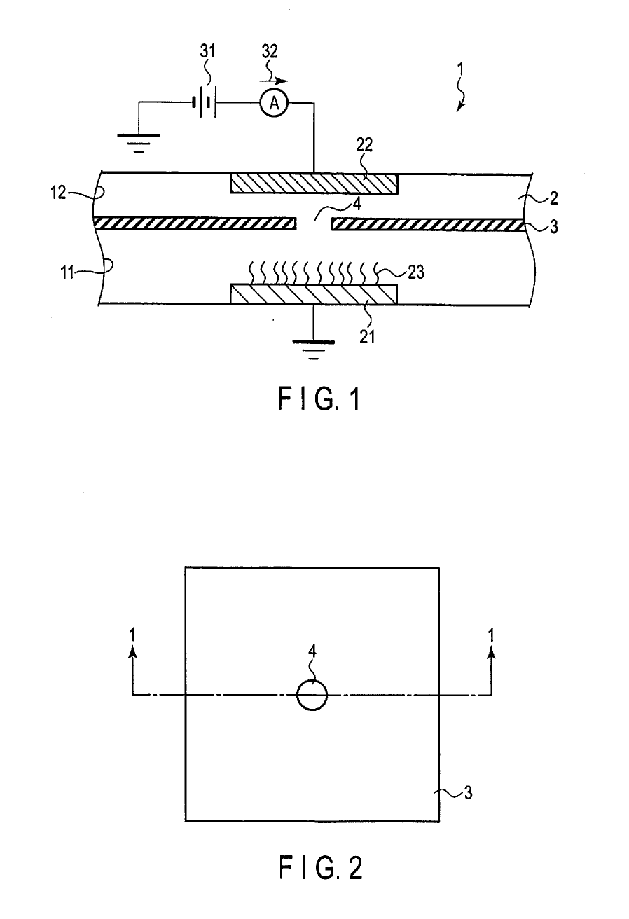

A shape of the-through hole 4 is, for example, a circle as shown in a plan view of FIG. 2. The partition 3 shown in FIG. 1 corresponds to a cross-sectional view along line 1-1 of FIG. 2. When the influenza virus having about 100 nm size is to be detected as the detection target, a diameter of the through hole 4 is, for example, 200-500 nm. The diameter of the influenza virus ranges between 80 and 120 nm in general, and thus, the diameter of the through hole 4 is, preferably, set to 200 to 300 nm in order to improve the detection sensitivity, for example.

The first chamber 11 is configured to be supplied with a sample liquid (first liquid) which is not shown, and the inside of the first chamber 11 can be filled with the sample liquid.

The sample liquid is an electrically conductive liquid containing a sample. The sample liquid is, for example, a liquid including the sample and a buffer solution, or a liquid including the sample and an electrolyte solution. The sample is collected from, for example, a biological body such as animal including human. The sample may possibly contain the detection target, and thus the sample liquid also may possibly contain the detection target.

The buffer solution includes, for example, phosphate buffered saline (PBS), tris-buffered saline (TBS), tris Ethylene diamine tetra acetic acid (TE), or 4-(2-hydroxyethyl)-1-piperazineethanesulfonic acid (HEPES). The electrolyte solution is, for example, KCl aqueous solution. The buffer solution and the electrolyte solution have pH of approximately 7 to 8. When such a buffer solution is used, the influenza virus is negatively-charged in the sample liquid.

The second chamber 12 is configured to be supplied with non-sample liquid (second liquid) which is not shown, and the inside of the second chamber 12 can be filled with the non-sample liquid. The non-sample liquid is a conductive liquid which does not contain the sample, and is, for example, a buffer solution containing PBS or TE, or electrolyte solution such as a KCl aqueous solution.

A lower elected (first electrode) 21 is provided in the first chamber 11. More specifically, the lower electrode 21 is provided on the lower surface in the first chamber 11. The through hole 4 of the partition 3 is positioned above the lower electrode 21.

A probe 23 is provided on an upper surface of the lower electrode 21, and binds specifically to the detection target. The probe 23 is connected to (fixed on) the lower electrode 21 before using the detection device 1 (before executing the detection method), but the probe 23 is detached from the lower electrode 23 during use of the detection device 1 (during execution of the detection method).

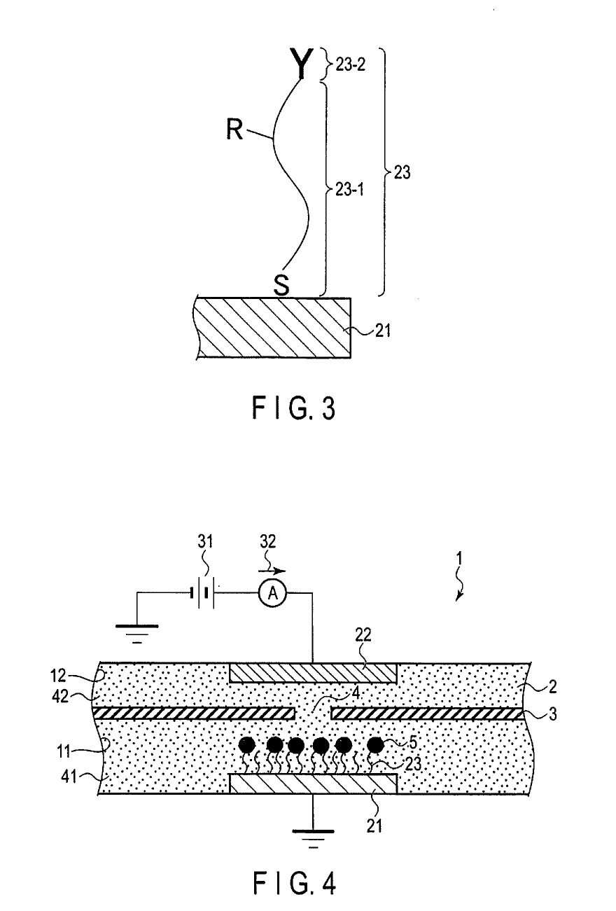

FIG. 3 is a view schematically depicting the probe 23 connected to the lower electrode 21.

The probe 23 includes a first part 23-1 connected to the lower electrode 21. In FIG. 3, the first part 23-1 is thiol (R—SH) having a functional group of —SH, where R, S, and H represent organic group, sulfur, and hydrogen, respectively. Hydrogen (not shown) of thiol is bound with the lower electrode 21 (covalent bound).

Instead of thiol, disulfide or thiocyanate may be used. In addition to an organic compound containing sulfur such as thiol or disulfide, a chemical compound containing organoselenium, organotellurium, isocyanide, isocyanate, or alkylsilane may be used.

As shown in FIG. 35, when an end 10 of the probe 23 is a functional group reactive to carbonic acid, amine, alcohol, or the like, one end 20a of a different probe 23′ can be bound to the end (functional group) 10, and the other end 20b of the probe 23′ can be detachably connected to the lower electrode 21.

When the probe 23 contains protein, the probe 23 can be detachably connected to a member containing, for example, mica, silica, or glass (a member different from the lower electrode 21) by using imprinting technique. In that case, the probe 23 is not detachably connected to the lower electrode 21, however the lower electrode 21 is required for the electrophoresis (electric field), as will be described later.

When the probe 23 contains DNA, the probe 23 can be detachably connected to, for example, a Si₃N₄ substrate (substrate mainly containing silicon nitride).

When the probe 23 is connected to the lower electrode 21, the material of the lower electrode 21 is selected such that the first part 23-1 can be detachably connected to the lower electrode 21. In the present embodiment, the material of the lower electrode 21 contains gold (Au); one of the materials with which thiol can be combined. The surface of the lower electrode 21 contains gold; however, the entirety of the lower electrode 21 is not necessarily gold. Instead of gold, a material such as silver (Ag), copper (Cu), mercury (Hg), or platinum (Pt) can be used.

Note that the orientation of the lower electrode 21 may be set such that the first part 23-1 can be detachably connected to the lower electrode 21.

As shown in FIG. 3, the probe 23 further includes a second part 23-2 which binds specifically to a detection target. The second part 23-2 is connected to the end of the first part 23-1 opposite to the lower electrode 21 side. The second part 23-2 contains, for example, antibody, nucleic acid, peptide, or chain of sugar.

Foreign substances in the sample liquid may be absorbed by the probe 23 in a non-specific manner. Thus, a material which can suppress non-specific absorption of foreign substances may be added to the probe 23. The material may contain, for example, polyethylene glycol chain with molecular mass of 100 to 100000.

Referring back to FIG. 1, an upper electrode (second electrode) 22 is provided in the second chamber 12. More specifically, the upper electrode 22 is provided on the upper surface in the second chamber 12. The through hole 4 of the partition 3 is positioned below the upper electrode 22. The upper electrode 22 is disposed to face the lower electrode 21. The material of the upper electrode 22 is, for example, silver or silver chloride (AgCl). The material of the upper electrode 22 may be platinum or gold.

The detection device 1 further includes a direct current power supply 31 and a measurement circuit (measurement device) 32 which are connected in series with respect to the upper electrode 22.

Now, a detection method using the detection device 1 will be explained.

Firstly, as shown in FIG. 4, the first chamber 11 is filled with a sample liquid 41 and the second chamber 12 is filled with a non-sample liquid 42. The lower electrode 21 is immersed in the sample liquid 41 and the upper electrode 22 is immersed in the non-sample liquid 42. The following description is given that the sample liquid 41 contains the detection target 5.

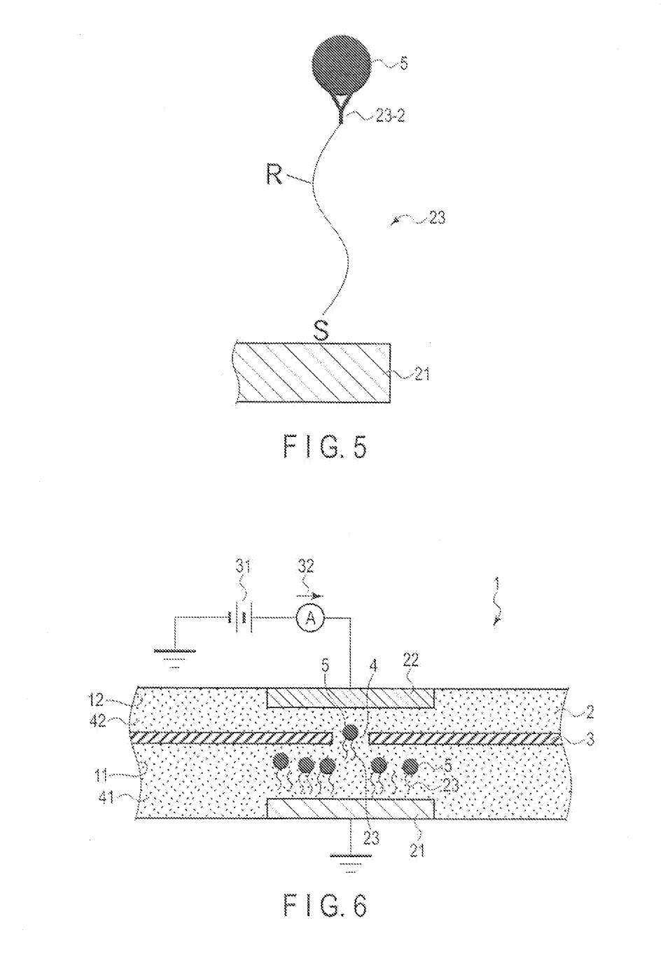

The detection target 5 combines with the probe 23. More specifically, as shown in FIG. 5, the detection target 5 binds specifically to an end of the second part 23-2 of the probe 23.

Note that, in FIGS. 4 and 5, the detection target 5 is depicted as a particle; however, the detection target 5 may not be a particle when being depicted in a further enlarged manner.

Subsequently, a voltage is applied between the lower electrode 21 and the upper electrode 22 by the direct current power source 31. In the present embodiment, a potential of the upper electrode (V2) is set greater than a potential of the lower electrode 21 (V1) (V1<V2). The sample liquid 41 and the non-sample liquid 42 are both conductive, and thus a current flows from the upper electrode 22 to the lower electrode 21.

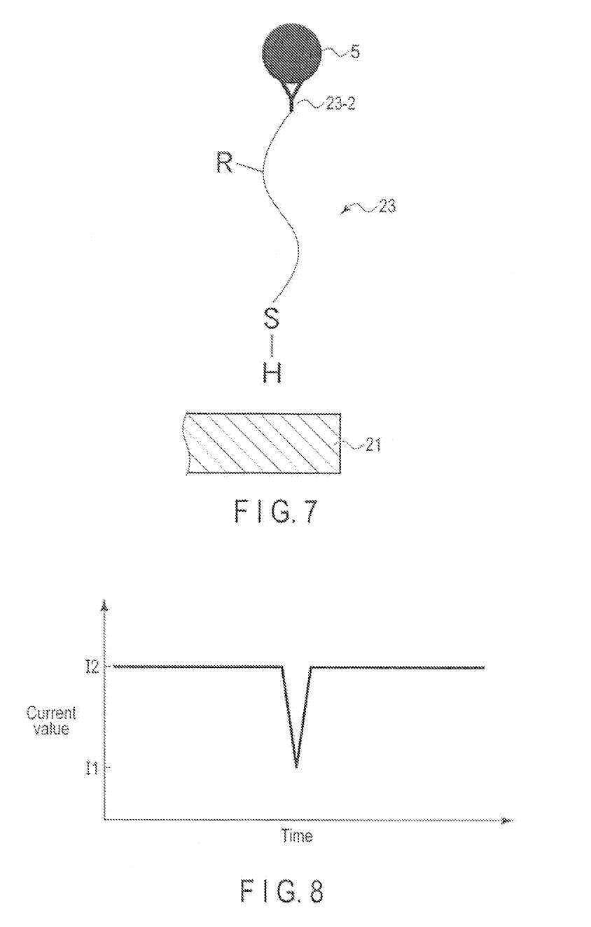

As a result, as shown in FIG. 6, the probe 23 is detached from the lower electrode 21. Specifically, as shown in FIG. 7, the first part (thiol) of the probe 23 connected to the lower electrode 21 (gold) is reduced (gold-thiol reduction reaction), and the probe 23 is detached from the lower electrode 21. As a result, the detection target 5 bound to the probe 23 is detached from the lower electrode 21. In the following description, the detection target 5 bound to the probe 23 may simply be referred to as detection target 5.

The detection target 5 in the sample liquid 41 in the first chamber 11 (that is, negatively charged influenza virus in this example) passes the fine hole 4 (liquid path) by electrophoresis (electric field), and moves to the non-sample liquid 42 in the second chamber 12.

Note that, if the detection target 5 is positively charged, V1>V2 is set by the direct current power source 31. As a result, the detection target 5 in the sample liquid 41 in the first chamber 11 passes the fine hole 4 (liquid path) by electrophoresis (electric field), and moves to the non-sample liquid 42 in the second chamber 12. In this case, it is possible to use a probe 23 capable of being detached from the lower electrode 21 under the V1>V2 condition.

When the detection target 5 is positively charged, and the probe 23 configured to be detached from the lower electrode 21 under V1<V2 condition, is used, another power supply different from the direct current power supply 31 is used to set the condition V1<V2, for example. Thereafter, the V1>V2 condition is set by the direct current power supply 31, and the detection target 5 in the sample liquid 41 in the first chamber 11 is moved to the second chamber 12 by electrophoresis (electric field).

When the direct current power source 31 is a variable power source, a condition of V1<V2 is set to detach the probe 23 from the lower electrode 21, and then, a condition of V1>V2 is set to move the detection target 5 in the first chamber 11 into the second chamber 12.

Referring back to FIG. 6, when the detection target 5 passes the fine hole 4 a current (current signals) measured by the measurement circuit 32 changes, for example, in a pulse shape as shown in FIG. 8, and a value of the current signal reduces. The reason is as follows.

When the detection target 5 is not passing through the fine hole 4, the number of ions in the fine hole 4, which is the current path between the lower electrode 21 and the upper electrode 22 is substantially constant, and thus, a substantially constant current signal (I2) flows between the lower electrode 21 and the upper electrode 22. That is, when the detection target 5 is not passing through the fine hole 4, a conductive state (electric state) between the lower electrode 21 and the upper electrode 22 is substantially constant.

On the other hand, when the detection target 5 passes the fine hole 4, the number of ions in the fine hole 4, which is current path between the lower electrode 21 and the upper electrode 22 is reduced by the detection target 5. As a result, a current resistance in the fine hole 4 increases, and the current signal decreases. That is, when the detection target 5 passes the fine hole 4, the conductive state between the lower electrode 21 and the upper electrode 22 changes. Then, after the detection target 5 passes the hole, the current signal returns to its original value 12, and the conductive state between the lower electrode 21 and the upper electrode 22 becomes substantially constant.

Therefore, it is determined whether the sample liquid 41 contains the detection target 5 or not, based on a presence or absence of the change of conduction condition (change of the electric state) between the lower electrode 21 and the upper electrode 22. The details will be explained below.

The value of current signal when the detection target 5 passes the fine hole 4 changes depending on the size, shape (steric structure), and surface state of detection target 5. Therefore, it is determined whether the sample liquid 41 contains the detection target 5 or not by comparing the measured current value with a previously obtained reference which is reduction of a current signal corresponding to the detection target 5.

For example, the size of an influenza virus which is the detection target 5 is basically well-known. Thus, only a reduction of current signal within a certain range can be regarded as a signal derived from the influenza virus, and others can be regarded as a signal derived from foreign substances.

The measurement circuit 32 has the above determination function, for example. That is, the measurement circuit 32 may be determination means. Alternately, a different device may have the above determination function instead of the measurement circuit 32. In that case, the determination means includes both the measurement circuit 32 and the device.

Since only a single detection target 5 passing through the fine hole 4 can indicate a presence of the detection target 5, the minimum detection sensitivity of the detection device 1 is high. Thus, in the present embodiment, the detection sensitivity can be improved.

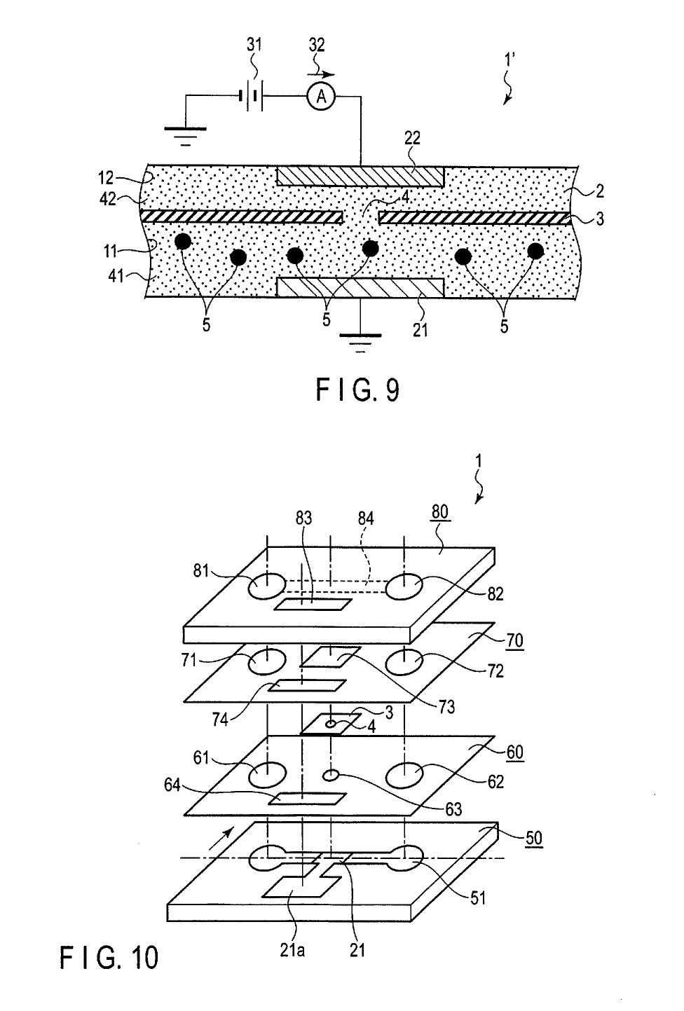

Here, as shown in FIG. 9, with respect to a detection device without the probe on the lower electrode 21 (comparative detection device 1′), the detection targets 5 diffuse in the sample liquid 41. Thus, with the same concentration of detection targets 5 in the sample liquid 41, the concentration of detection targets 5 in the vicinity of the fine hole 4 of the detection device 1′ of comparative example is lower than that of the detection device 1 of the embodiment.

In general, the lower the concentration of the detection targets 5 in the vicinity of the fine hole 4, the lower the possibility that the detection targets 5 pass through the fine hole 4 in a predetermined time, and the longer time requires to detect the detection target 5.

In a detection method using the detection device 1′ (detection method of comparative example), a long time is required to detect a detection target 5 unless the concentration of detection targets 5 in the first chamber 11 is 1×10⁷/mL or more.

On the other hand, in the detection method using the detection device 1 (detection method of the present embodiment), even if the concentration of detection targets 5 in the first chamber is less than 1×10⁷/mL, the concentration of detection targets 5 in the vicinity of the fine hole 4 becomes greater than 1×10⁷/mL by virtue of the probe 23. As a result, the detection method of the present embodiment can rapidly detect a detection target 5 even with the sample liquid 41 having such a low concentration of detection targets 5 that the detection has been difficult. As can be understood from the above, in the present embodiment, not only improvement of the detection sensitivity but also reduction of the detection time can be achieved.

In general, as the density of the probes 23 increases, the effect of the present embodiment (reduction of the detection time) increases. Furthermore, the density of probes 23 may not be uniform. For example, the density of probes 23 may be maximized right below the fine hole 4 and in the vicinity thereof.

Note that, when the detection target 5 does not exist in the sample liquid 41, the current measured by the measurement circuit 32 does not change. Thus, when the change in the current signals (detection signal) is not detected in the measurement which is performed a predetermined period of time, it is determined that the sample liquid 41 does not contain the detection target 5.

Furthermore, if a single detection target 5 passes the through hole 4, a single pulse-like current signal reduction (detection signal) is generated. Thus, for example, on the basis of the number of detection signals in a certain period of time, the number of detection targets 5 in the sample liquid 41 can be estimated. From this estimated number, for example, whether or not the infection stage is early can be determined.

Note that, in the present embodiment, the configuration to measure the current value by the measurement circuit 32 is employed, however, a configuration to measure voltage by the measurement circuit 32 may be employed. In this case, the presence or absence of the detection target is determined on the basis of a change in voltage signals measured by the measurement circuit 32 (detection signal).

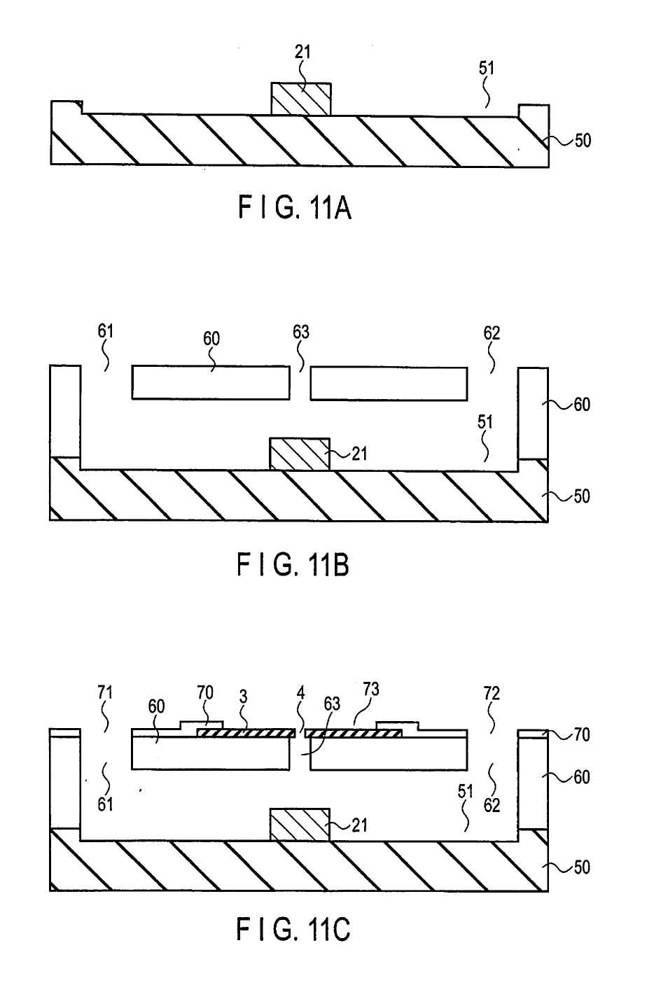

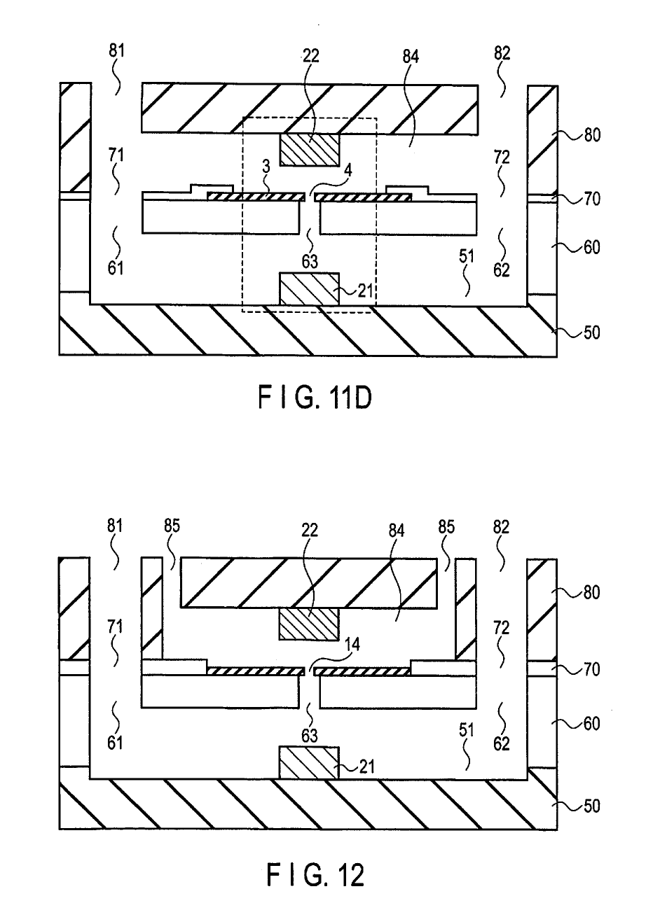

Now, an example of a manufacturing method of the detection device 1 of the present embodiment will be explained with reference to FIG. 10 and FIGS. 11A to 11D.

FIG. 10 illustrates an exploded perspective view of the detection device 1 of the present embodiment. FIGS. 11A to 11D are cross-sectional views of a manufacturing method of the detection device 1. FIGS. 11A to 11D are cross-sectional views taken along a line with single-dots of FIG. 10, as being viewed orthogonally to the arrow.

A groove 51 is formed on the surface of a first substrate 50 (FIGS. 10 and 11A). The surface of the first substrate 50 does not possess conductivity. The material of the first substrate 50 is an insulator which is, for example, glass, resin such as polydimethylsiloxane (PDMS), or silicon oxide (SiO₂).

The groove 51 is used as a liquid path through which a liquid flows. The groove 51 includes, in a top view, two circular groove areas and a rectangular groove area connecting therewith (FIG. 10). The diameter of the circular groove areas is greater than a short side of the rectangular groove area. The groove 51 may be formed by using chemical treatment such as etching process or physical treatment such as curving.

Then, the lower electrode 21 is formed on the first substrate 50 (FIGS. 10 and 11A), and an extraction electrode 21a of the lower electrode 21 is formed (FIG. 10). The lower electrode 21 is formed in the groove 51. A process to form the lower electrode 21 and the extraction electrode 21a includes forming a conductive film using sputtering or evaporation, and etching the conductive film.

Then, the probe 23 shown in FIG. 1 is formed on the lower electrode 21.

Note that the probe 23 is omitted in FIGS. 10 and 11A to 11F for the simplification. The formation of probe 23 is performed by, for example, supplying a solution containing probes 23 to the lower electrode 21 while the extraction electrode 21a is masked by a resist or the like. The functional group (—SH) of the probe 23 in the supplied solution is connected to the lower electrode 21. Then, the mask is remove. As a result, the structure including the lower electrode 21 and the probe 23 connecting thereto is obtained. That is, the electrode with probe used in the detection device which, contributes to the improvement of the detection sensitivity and the reduction of the detection time, is obtained.

A first packing (first spacer) 60 is provided on the first substrate 50 (FIGS. 10 and 11B). The first packing 60 is provided with through the holes 61, 62, and 63 shown in FIG. 11B and the through hole 64 shown in FIG. 10.

Through holes 61 and 62 are positioned above the two circular groove areas. Through holes 61 and 62 are paths which introduce or collect the liquid into or from the groove 51. Through hole 63 is positioned above the lower electrode 21. Through hole 64 is positioned above the extraction electrode 21a.

The first packing 60 is formed of, for example, an adhesive resin such as silicon rubber or PDMS, combination of a resin such as polyethylene terephthalate (PET) film and an adhesive agent applied thereon, or a combination of a resin such as PET film and a hydrophilic film applied thereto. With such a material, the first packing 60 is fixed on the first substrate 50. The first packing 60 may be fixed on the first substrate 50 using an adhesive agent or an adhesive layer which is not shown. A forming method of the first packing 44 includes processing a member containing the above material by laser.

Subsequently, the partition 3 is provided on the first packing 60, and then, a second packing (second spacer) 70 is provided on the first packing 60 (FIGS. 10 and 11C).

The second packing 70 is structured to cover an upper surface of an edge of the partition 3, and the edge of the partition 3 is hold between the first packing 60 and the second packing 70 and thereby being fixed. The thickness of the second packing 70 is set to be thicker than the depth of the through hole 4 of the partition 3, for example.

The second packing 70 is provided with the through holes 71, 72, and 73 shown in FIG. 11C and the through hole 74 shown in FIG. 10.

Through holes 71 and 72 are positioned above through holes 61 and 62. The through holes 71 and 72, and together with the through holes 61 and 62 are used as the flow path to introduce or collect the liquid into or from the groove 51. The through hole 73 is formed to be positioned on the partition 3, and the through hole 74 is formed to be positioned above the extraction electrode 21a.

A material of the second packing 70 is, for example, same as the material of the first packing 60 mentioned above. By using such a material, the second packing 70 can be fixed on the first packing 60. In addition, the second packing 70 may be fixed on the first packing 60 by using an adhesive compound or adherent layer (not shown). The first packing and second packing may further have adhesiveness to the partition 3. A forming method of the second packing 70 is same as the forming method of the first packing 60.

The first substrate 50, first packing 60, and second packing 70 define the first chamber 11 of FIG. 1.

Through holes 81, 82, and 83 and groove 84 are formed in a second substrate 80 (FIG. 10). The groove 84 is on the rear side of the second substrate 80, and depicted by in a dotted line on FIG. 10. A material of the second substrate 80 is insulator, for example, resin that does not have conductive property such as polymethyl methacrylate, or glass. A method of forming the through holes 81, 82, 83 and the trench 84 is performed by, for example, applying laser processing, or physical process such as excavating to a member containing the above material.

After that, as shown in FIG. 11D, the upper electrode 22 is formed in the groove 84, and the second substrate 80 is provided on the second packing 70 in a manner that the upper electrode 22 faces the second packing 70. When the second packing 70 has adhesiveness, the second substrate 80 can be fixed on the second packing 70. The second substrate 80 may be fixed on the second packing 70 by using an adhesive compound or adherent layer.

The through holes 81, and together with the through holes 71, 72, 61 and 62 are used as the flow path to introduce or collect the liquid into or from the trench 51 (FIG. 11D).

The second packing 70 and the second substrate 80 define the second chamber 12 of FIG. 1. The detection device of FIG. 1 corresponds to an area surrounded by the dotted line of FIG. 11D.

In FIG. 11D, for example, after the first chamber is filled with the sample liquid by supplying the sample liquid from the through holes 81, 71 and 61 (and/or through holes 82, 72 and 62), the second chamber can be filled with the non-sample liquid by supplying the non-sample liquid from the through holes 81, 71 and 61 (and/or through holes 82, 72 and 62).

Note that, as shown in FIG. 12, by forming a second substrate 80 including another through hole 85 for supplying the non-sample liquid in addition to the through holes 81, 71 and 61 for supplying the sample liquid, a mixture of the sample liquid and the non-sample liquid can be suppressed.

As can be understood from the above, the present embodiment allows provide the detection device, the detection method, and the electrode with the probe which are advantageous to increase the detection sensitivity and reduce the detection time, by employing the configuration to increase the concentration of the detection targets 5 in the vicinity of the fine hole 4 by using the probe 23 provided on the lower electrode 21.

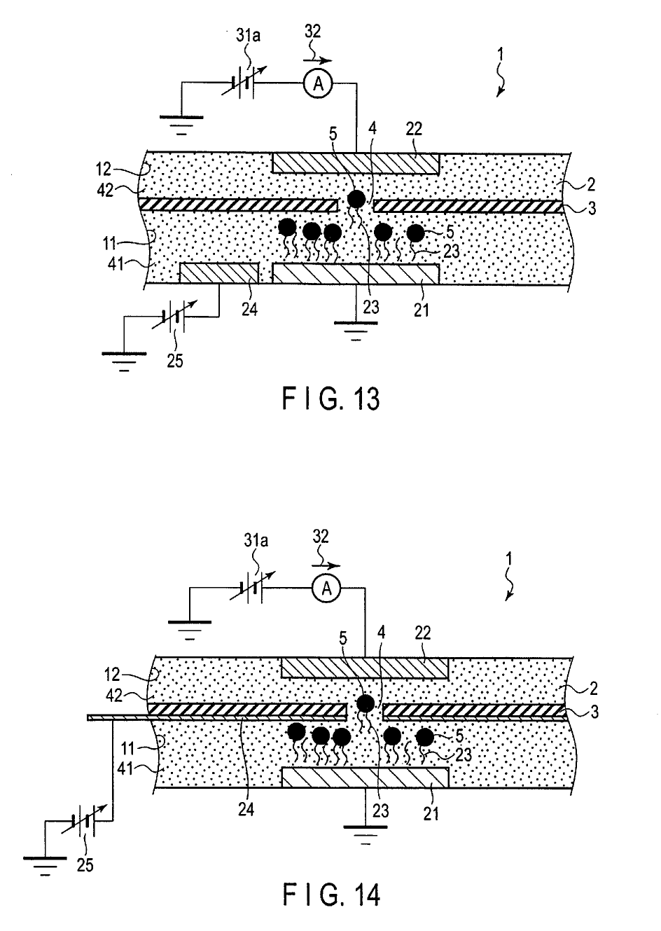

FIG. 13 is a view schematically depicting a detection device according to a first variation of the present embodiment.

In the first variation, a variable power source 31a is used instead of the direct current power source 31, an electrode (seventh electrode) 24 is provided in the first chamber 11, and a variable power source 25 is provided to supply a voltage to the electrode 24. The electrode 24 is used to detach the probe 23 from the lower electrode 21. A material of the electrode 24 is, for example, Pt. In the following description, the electrode 24 will be referred to as the separation electrode 24. Note that, in FIG. 13, the separation electrode 24 is smaller than the lower electrode 21; however, the size of the separation electrode 24 may be equal to the size of the lower electrode 21.

In the first variation, for example, the probe 23 is detached from the lower electrode 21 as follows. A potential (V2) of the upper electrode 22 is set to zero by the variable power source 31a, and a potential (V3) of the separation electrode 24 is set greater than a potential of the lower electrode 21 (V1) by the variable power source 25. Since the sample liquid 41 is conductive, a current flows from the separation electrode 24 to the lower electrode 21, and the probe 23 is detached from the lower electrode 21.

Note that, when the detection target 5 is negatively charged, the probe 23 can be detached from the lower electrode 21 by setting V3>V1, V2, in general. Thus, V2 is not necessarily zero. However, in consideration of power consumption, V2 is, preferably, set to zero.

Furthermore, in the first variation, for example, the detection target 5 in the first chamber 11 is moved into the second chamber by electrophoresis as follows. When the detection target 5 is negatively charged, the potential (V2) of the upper electrode 22 is set greater than the potential (V1) of the lower electrode 21 by the variable power source 31a, and the potential (V3) of the separation electrode 24 is set to zero by the variable power source 25.

Note that, when the detection target 5 is negatively charged, the detection target 5 in the first chamber 11 can be moved into the second chamber 12 by electrophoresis by setting V3>V1, V2, in general. Thus, V2 is not necessarily zero. However, in consideration of power consumption, V2 is, preferably, set to zero.

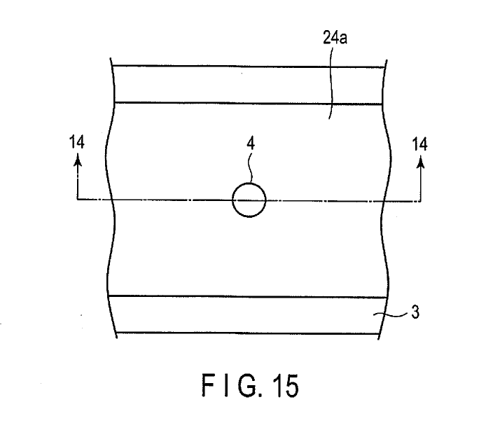

FIG. 14 is a view schematically depicting a detection device according to a second variation of the present embodiment. FIG. 15 is a plan view illustrating a partition and a separation electrode of the detection device of the second variation.

In the first variation, the separation electrode 24 is provided on the lower surface side of the first chamber 11, whereas in this second variation, the separation electrode 24 is provided with the upper surface side of the first chamber 11. In FIG. 14, the separation electrode 24 is provided on the partition 3 in the first chamber 11. In the second variation, when the detection targets are moved by electrophoresis, the potential difference between the electrodes 21 and 24 may be used.

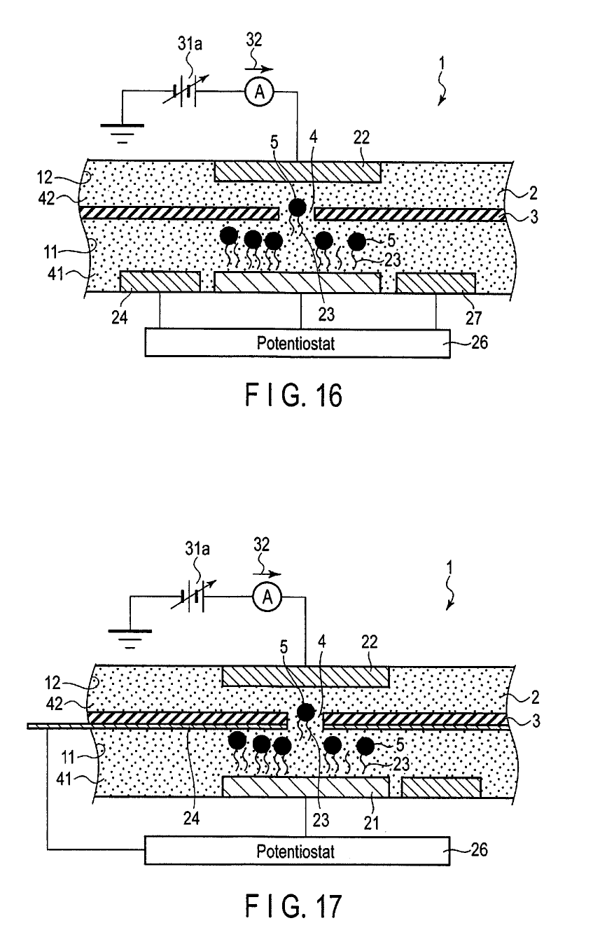

FIG. 16 is a view schematically depicting a detection device of a third variation of the present embodiment.

The third variation is different from the first variation in that the third variation further comprises a three-electrode system potentiostat 26, and a reference electrode 27. The potentiostat 26 has a reference pole, an action pole, and an opposing pole. The reference electrode 27 is disposed in the first chamber 11. A material of the reference electrode 27 is, for example, AgCl.

The reference electrode 27, the separation electrode 24, and the lower electrode 21 are connected to the reference pole, the action pole, and the opposite pole of the potentiostat 26, respectively. By using the potentiostat 26, it is possible to accurately control the voltage between the lower electrode 21 and the separation electrode 24. This enables an accurate separation of the lower electrode 21 from the probe 23.

FIG. 17 is a view schematically depicting a detection device according to a fourth variation of the present embodiment.

In the fourth variation, the potentiostat 26 is added to the second variation, and the separation electrode 24 is connected to the opposition pole of the potentiostat 27.

### Second Embodiment

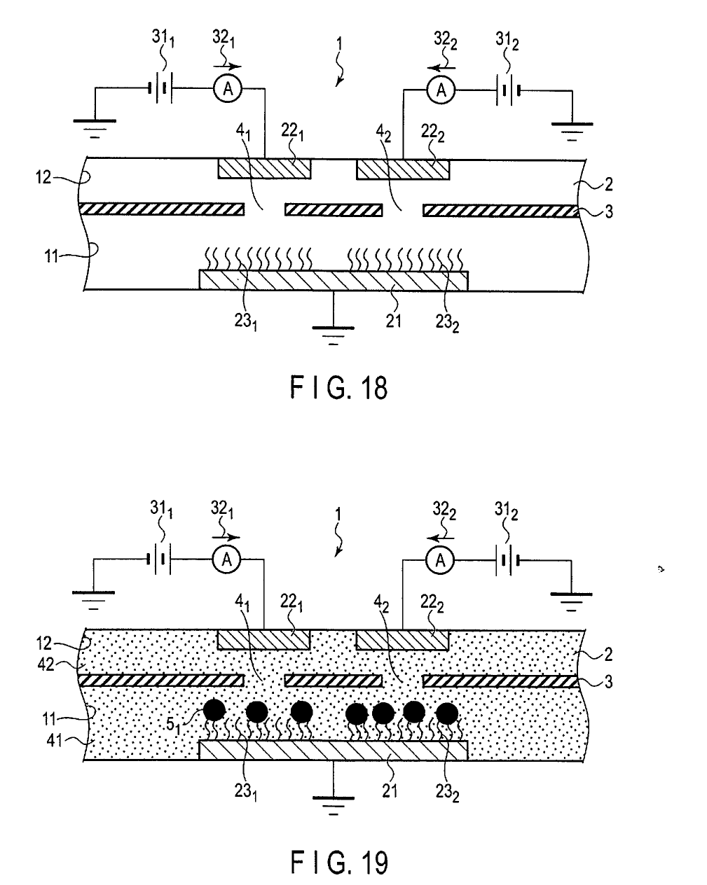

FIG. 18 is a view schematically depicting a detection device according to the second embodiment.

The present embodiment is different from the first embodiment in that two different kinds (plural kinds) of detection targets can be detected. In order to detect the two kinds of the detection targets, the detection device 1 of the present embodiment comprises two probes 23₁ and 23₂, two through holes 4₁ and 4₂, two upper electrodes 22₁ and 22₂, two direct current power supply 31₁ and 31₂, and two measurement circuit 32₁ and 32₂.

The probe 23₁ (hereafter referred to as a first probe 23₁) binds specifically to a first detection target, the probe 23₂ (hereafter referred to as a second probe 23₂) binds specifically to a second detection target which is different from the first detection target.

The through hole 4₁ has a size (diameter) corresponding to the first detection target, and the through hole 4₂ has a size (diameter) corresponding to the second detection target. Hereafter, the through hole 4₁ and 4₂ may be described as fine holes 4₁ and 4₂, respectively.

The through hole 4₁ is positioned below the upper electrode 22₁. In addition, the through hole 4₂ is positioned below the upper electrode (fourth electrode) 22₂. The upper electrodes 22₁ and 22₂ are disposed to face the lower electrode 21.

The direct current power supply 31₁ and the measurement circuit 32₁ are connected with the upper electrodes 22₁ in series. Similarly, the direct current power supply 31₂ and the measurement circuit 32₂ are connected with the upper electrodes 22₂ in series.

A potential of the direct current power supply 31₁ may be same as or different from a potential of the direct current power supply 31₂. That is, the potentials of the direct current power supplies 31₁ and 31₂ are selected such that the first detection target and the second detection target can be easily detected. In addition, when the potentials are the same, it is possible to employ a common direct current power supply as the two direct current power supplies 31₁ and 31₂. When the potentials are different, the direct current power supply 31₁ and the direct current power supply 31₂ may be replaced with a single variable power supply.

The electrode (21, 23₁, 23₂) with the probes of the present embodiment can be formed, for example, by the following process.

After the lower electrode 21 is formed, a solution containing the first probe 23₁ is supplied on a partial region of the lower electrode 21, and then a solution containing the second probe 23₂ is supplied on another partial region of the lower electrode 21. The supply of the solution containing the first probe 23₁, and the supply of the solution containing the second probe 23₂ are performed by, for example, using a screen printing technique, or an ink jet printing technique. By using such the technique, the solution containing the first probe 23₁ can be easily and selectively supplied on an arbitrary region of the lower electrode 21, and similarly the solution containing the second probe 23₂ can be easily and selectively supplied on an arbitrary region of the lower electrode 21.

Note that, the electrode (21, 23₁, 23₂) with the probes are also obtained by the forming method using the mask as in the first embodiment.

Now, a detection method using the detection device 1 of the present embodiment will be explained.

First, as shown in FIG. 19, the first chamber 11 is filled with the sample liquid 41, and the second chamber 122 is filled with non-sample liquid 42. The following description is given that the sample liquid 41 contains negatively charged detection targets 51 and 52.

Since the first prove 23₁ binds specifically to the first detection target 5₁; the concentration of the first detection target 5₁ is high in the vicinity of the fine through hole 4₁. Similarly, since the second prove 23₂ binds specifically to the second detection target 5₂, the concentration of the second detection target 5₂ is high in the vicinity of the fine through hole 4₂.

Subsequently, a voltage is applied between the lower electrode 21 and the upper electrode 22₁ by the direct current power supply 31₁, and a voltage is applied between the lower electrode 21 and the upper electrode 22₂ by the direct current power supply 31₂.

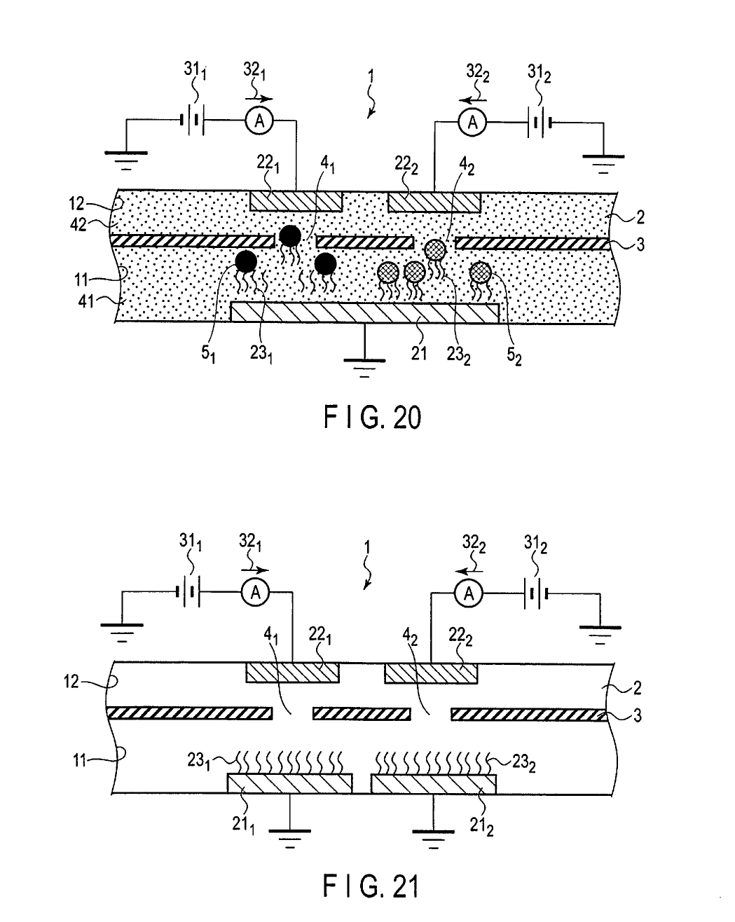

As a result, as shown in FIG. 20, the first prove 23₁ and the first detection target 5₁ combined therewith are detached from the lower electrode 21. Similarly, the second prove 23₂ and the second detection target 5₂ combined therewith are detached from the lower electrode 21. In the following description, the first detection target 5₁ bound to the first probe 23₁ and the second detection target 5₂ bound to the second probe 23₂ may be simply referred to as the first detection target 5₁ and the second target detection 5₂, respectively.

The first detection target 5₁ in the sample liquid 41 in the first chamber 11 moves into the non-sample liquid 42 in the second chamber 12 via the fine through hole 4₁ by electrophoresis (electric field). In response to the first detection target 5₁ passing through the fine hole 4₁, the conductive state between the lower electrode 21 and the upper electrode 22₁ changes. Since the concentration of the first detection targets 5₁ is high in the vicinity of the fine hole 4₁, the conductive state easily changes. Consequently, based on a time change of the current signal measured by the measurement circuit 32₁, it is possible to determine whether the sample liquid 4₁ contains the first detection target 5₁ in a short time.

The second detection target 5₂ in the sample liquid 41 in the first chamber 11 moves into the non-sample liquid 42 in the second chamber 12 via the fine through hole 4₂ by electrophoresis (electric field). In response to the second detection target 5₂ passing through the fine hole 4₂, the conductive state between the lower electrode 21 and the upper electrode 22₂ changes. Since the concentration of the second detection targets 5₂ is high in the vicinity of the fine hole 4₂, the conductive state easily changes. Consequently, based on a time change of the current signal measured by the measurement circuit 32₂, it is possible to determine whether the sample liquid 4₁ contains the second detection target 5₂ in a short time.

Note that, the time of applying the voltage between the lower electrode 21 and the upper electrode 22₁ may be different from the time of applying the voltage between the lower electrode 21 and the upper electrode 22₂. For example, when the potential to be applied to the lower electrode 21 is different from the potential to be applied to the upper electrode 22₁, and the direct current power sources 31 and 93 are replaced with the single variable power supply, the two voltages are applied at different times.

Further, in the present embodiment, as shown in FIG. 18, the plurality of first probes 23₁ are disposed on the left side of the lower electrode 21, and the plurality of the second probes 23₂ are disposed on the left side of the upper electrode 22. However, all of, or a part of the plurality of the first probes 23₁ and the plurality of the second probes 23₂ may be disposed to mix on the lower electrode 21.

Furthermore, the number of the plurality of the first probes 23₁ and the number of plurality of the second probes 23₂ may be the same or may be different. The dimensions of the upper electrode 22₁ and the dimensions of the upper electrode 22₂ may be same or may be different.

In accordance with the detection device and the detection method to detect two kinds of detection targets of the present embodiment, a detection device and a detection method to detect three or more detection targets can be achieved.

In addition, in the present embodiment, the first probe 23₁ and the second probe 23₂ are configured to bind specifically to the different kinds of the detection targets, respectively, however, the first probe 23₁ and the second probe 23₂ may be configured to bind specifically to the same kind of the detection target. That is, the detection device and the detection method of the present embodiment is also applicable to the detection device and the detection method which are directed to a single kind of detection target.

### Third Embodiment

FIG. 21 shows a schematic view of a detection device 1 of the third embodiment.

The present embodiment is different from the second embodiment in that the present embodiment comprises two (plural) lower electrodes 21₁ and 21₂.

The first probe 23₁ is detachably connected to the lower electrode 21₁, and the second probe 23₂ is detachably connected to the lower electrode (third electrode) 21₂.

The upper electrode 22₁ is disposed to face the lower electrode 21₁, and the upper electrode 22₂ is disposed to face the lower electrode 21₂.

A through hole 4₁ is positioned between the lower 10. electrode 21₁ and the upper electrode 22₁, and a through hole 4₂ is positioned between the lower electrode 21₂ and the upper electrode 22₂.

Now, a detection method using the detection device 1 of the present embodiment will be explained.

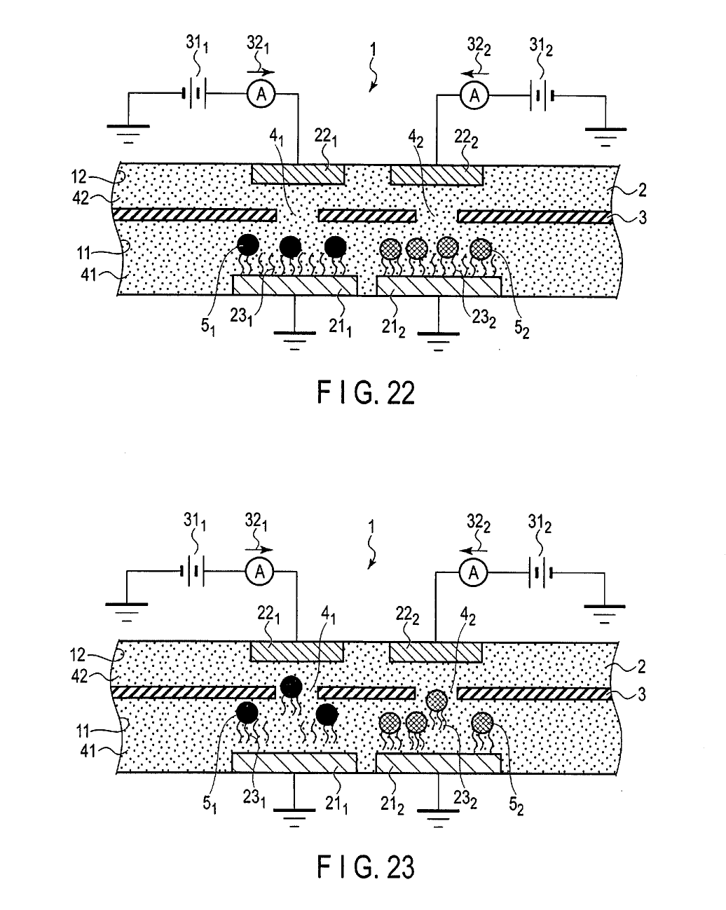

First, as shown in FIG. 22, the first chamber 11 is filled with the sample liquid 41, and the second chamber 12 is filled with the non-sample liquid 42.

Since the first probe 23₁ binds specifically to the first detection target 5₁, the concentration of first detection targets 5₁ becomes high in the vicinity of the fine hole 4₁. Similarly, since the second probe 23₂ binds specifically to the second detection targets 5₂, the concentration of second detection targets 5₂ becomes high in the vicinity of the fine hole 42.

Subsequently, a voltage is applied between the lower electrode 21₁ and the upper electrode 22₁ by using a direct current power supply 31₁, and a voltage is applied between the lower electrode 21₂ and the upper electrode 22₂ by using a direct current power supply 31₂.

As a result, as shown in FIG. 23, the first probe 23₁ and the first detection target 5₁ combined therewith are detached from the lower electrode 21₁. Similarly, the second probe 23₂ and the second detection target 5₂ combined therewith are detached from the lower electrode 21₂.

The first detection target 5₁ in the sample liquid 41 in the first chamber 11 moves into the non-sample liquid 42 in the second chamber 12 through the fine hole 4₁ by electrophoresis (electric field). When the first detection target 5₁ passes through the fine hole 4₁, a conductive state between the lower electrode 21₁ and the upper electrode 22₁ changes. Since the concentration of first detection targets 5₁ is high in the vicinity of the fine hole 4₁, the conductive state easily changes. Consequently, based on a time change of the current signal measured by the measurement circuit 321, it is possible to determine whether the sample liquid 41 contains the first detection target 5₁ in a short time.

The second detection target 5₂ in the sample liquid 41 in the first chamber 11 moves into the non-sample liquid 42 in the second chamber 12 through the fine hole 4₂ by electrophoresis (electric field). When the second detection target 5₂ passes through the fine hole 4₂, a conductive state between the lower electrode 21₂ and the upper electrode 22₂ changes. Since the concentration of second detection targets 5₂ is high in the vicinity of the fine hole 4₂, the conductive state easily changes. Consequently, based on a time change of the current signal measured by the measurement circuit 32₂, it is possible to determine whether the sample liquid 41 contains the first detection target 5₂ in short time.

In the present embodiment, the lower electrode 21₁ and the lower electrode 21₂ are physically different. Thus, the lower electrode 21₁ is easily optimized with respect to the first probe 23₁ and the lower electrode 21₂ is easily optimized with respect to the second probe 23₂. For example, it is possible to use the lower electrode 21₁ containing a material and/or having a plane direction which is suitable to easily detach the first probe 23₁ from the lower electrode 21₂, and the lower electrode 21₂ containing a material and/or having a plane direction which is suitable to easily detach the second probe 23₂ from the lower electrode 21₂.

A forming method of the lower electrodes 21₁ and 21₂ is, for example, same as the forming method of the lower electrode of the first embodiment.

As with the second embodiment, the detection device and the detection method of the present embodiment is applicable to a detection device and a detection method which are directed to detect three or more kinds of detection targets, and a detection device and a detection method which are directed to detect a single kind of detection target.

### Fourth Embodiment

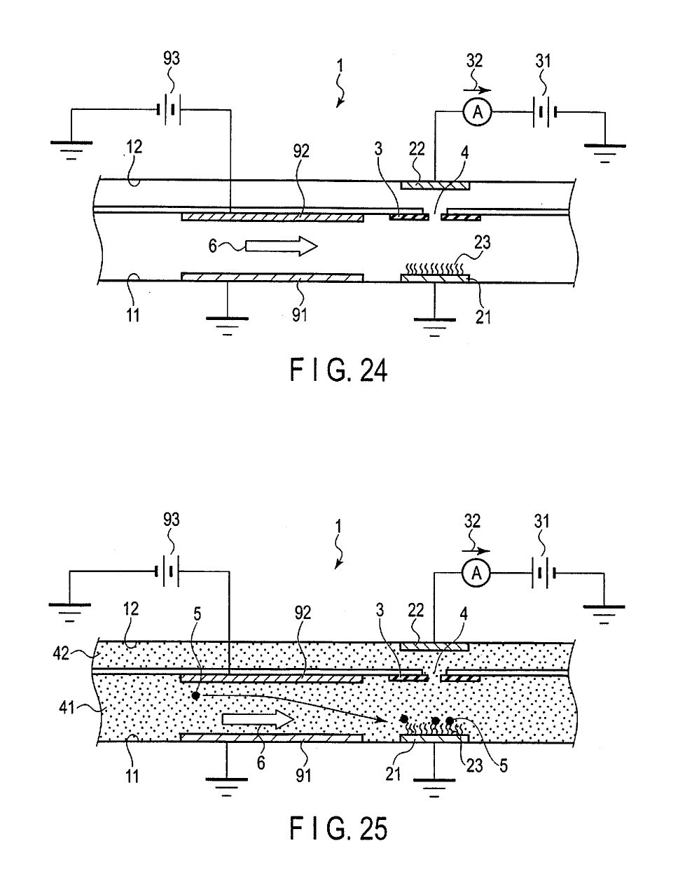

FIG. 24 is a view schematically depicting a detection device according to the fourth embodiment.

The detection device 1 of the present embodiment includes a first chamber 11 of a flow passage structure. A sample liquid (not shown) introduced into the first chamber 11 flows in a left-to-right direction 6, for example. As a result, the detection target (not shown) in the sample liquid flows in the left-to-right direction 6, thereby reducing time required for the detection target to combine with the probe 23. The flow passage structure of the present embodiment is applicable to the first to third embodiments, too.

The flow of the sample liquid can be generated by transferring liquid by using a pump (not shown). Alternatively, the flow of the sample liquid can be generated by providing a absorption band on the downstream (channel downstream) side of the sample liquid in the chamber 11 to cause the sample liquid to be absorbed in the absorption band. A flow speed of the sample liquid is appropriately changed in a range of 0.001 to 1000 μL/min, for example.

The detection device 1 of the present embodiment is different from the first to third embodiment further in that the detection device 1 includes a guide mechanism to guide the detection target in the sample liquid to the probe 23.

The guide mechanism includes a lower guide electrode (fifth electrode) 91, an upper guide electrode (sixth electrode) 92, and a direct current power supply 93. The lower guide electrode 91 is provided on a bottom surface of the first chamber 11 in a similar way as the lower electrode 21. The lower guide electrode 91 is grounded. The lower guide electrode 91 is disposed apart from the lower electrode 21 by a constant distance.

The upper guide electrode 92 is provided on the upper surface in the first chamber 11 and is connected to the direct current power source 93. The upper guide electrode 92 is disposed apart from the partition 3 by a constant distance such that the upper guide electrode 92 faces the lower guide electrode 91.

Note that, the direct current power supply 31 and the direct current power supply 31 may be changed to a single variable power supply. A material of the lower guide electrode 91 and a material of the upper guide electrode 92 are, for example, same as the material of the lower electrode 21.

Now, a detection method using the detection device 1 of the present embodiment will be explained.

First, as shown in FIG. 25, the second chamber 12 is filled with the non-sample liquid 42, and the first chamber 11 is filled with the sample liquid 41. The following description is given that the sample liquid 41 contains negatively charged detection targets 5.

A positive potential is applied to the lower guide electrode 91 and a negative potential is applied to the upper guide electrode 92 by using the direct current power source 93. As a result, electrical flux lines are generated from the lower guide electrode 91 to the upper guide electrode 92. At that time, the direct current power source 31 is off, and there is not a potential difference between the lower electrode 21 and the upper electrode 22.

Since the detection target 5 is negatively charged, downward force due to the electrical flux lines acts on the detection target 5 moving from left to right. As a result, the direction of movement of the detection target 5 is changed such that the detection target 5 moves closer to the probe 23 on the lower electrode 21.

In this way, by guiding the detection target 5 to come closer to the probe 23 (guiding process), the status shown in FIG. 4, that is, the detection target 5 is bound to the probe 23, can be easily achieved. After the guiding process is performed for a predetermined period of time, the direct current power supply 93 is switched off and the flow of the sample liquid 41 is stopped.

After that, similarly to the first embodiment, the voltage is applied between the lower electrode 21 and the upper electrode 22 by the direct current power supply 31 to detached the probe 23 from the lower electrode 21, and the determination of the presence or absence of the detection target 5 is performed by the measurement circuit 32.

FIG. 29 is a view schematically depicting a detection device of a variation of the present embodiment. In this variation, the upper guide electrode 92 is provided in the second chamber 12. More specifically, the upper guide electrode 92 is provided on the lower surface (bottom) in the second chamber 12.

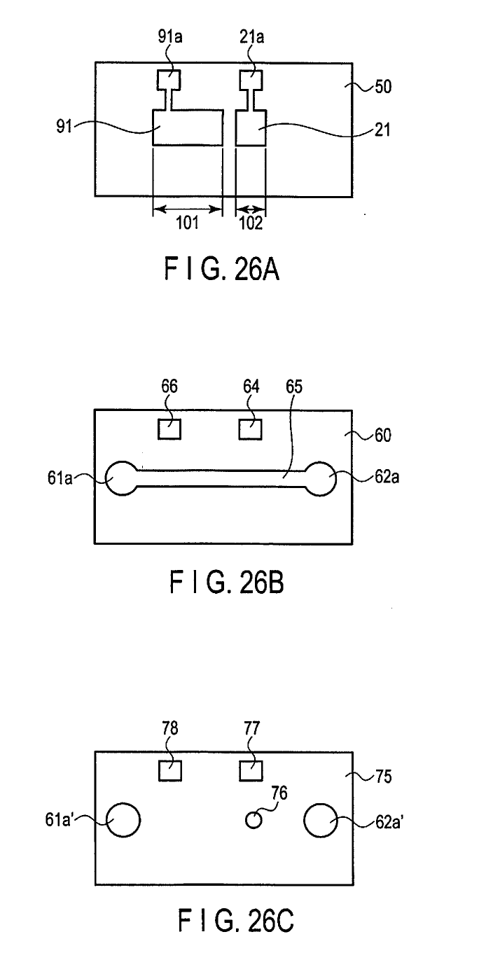

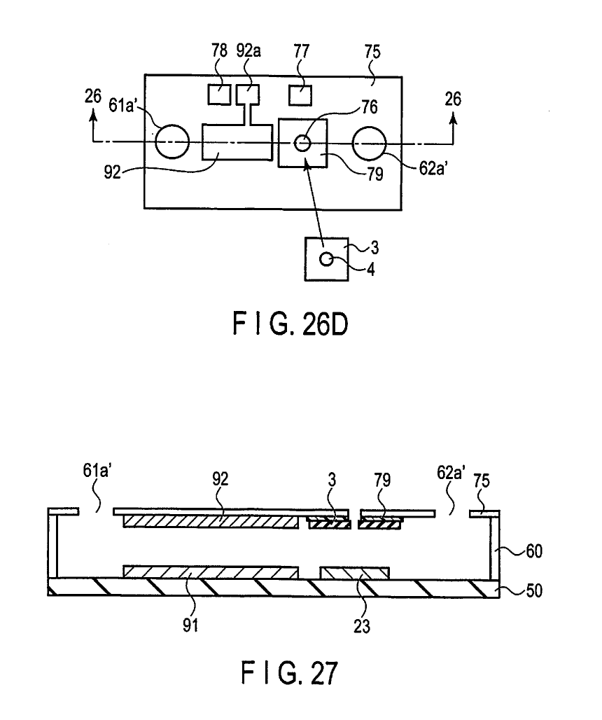

The lower surface in the second chamber 12 (upper surface in the first chamber) is defined by the film 75 shown in FIG. 26C. Since the film 75 is thin, a potential difference can be applied between the lower guide electrode 91 and the upper guide electrode 22 by the direct current power supply 93. As a result, the electrical flux lines mentioned above are generated, and the detection targets can be guided the near the probe 23.

In addition, the sample liquid is not flown in the second chamber 12, and thus the probe 32 is not required to be provided on the upper guide electrode 92 in the second chamber 12.

FIGS. 26A to 26D are plan views for explaining a manufacturing method of the detection device 1 of the present embodiment.

First, as shown in FIG. 26A, the lower electrode 21 and the extraction electrode 21a thereof, and the lower guide electrode 91 and a extraction electrode 91a thereof are formed on the first substrate 50. The first substrate 50 defines the bottom surface (bottom of the flow passage) of the first chamber 11.

A length 101 of the lower guide electrode 91 is, for example, 5 mm, and a length 102 of the lower electrode 21 is, for example, 1 mm. The lower guide electrode 91 and the lower electrode 21 have a width of, for example, 1 mm.

Subsequently, as shown in FIG. 26B, a packing (spacer) 60 is formed. The packing 60 defines a side surface (flow passage wall) of the first chamber 11. In the present embodiment, the packing 60 is given adherence.

The packing 60 is provided with through holes 61a and 62a corresponding to the through holes 61 and 62 (FIGS. 10 and 11B) of the first embodiment. The packing 60 is further provided with a through hole 65 corresponding to the flow passage ,and a through hole 66 to extract the extraction electrode 91a. The through hole 65 is formed to connect the through hole 61a and the through hole 62a, and the through holes 61a, 62a and 65 constitute a single through hole. A width of the through hole 65 defining the flow passage width is, for example, 1 mm, and the thickness of the packing 60 defining the flow passage height is, for example, 25 μm.

Subsequently, as shown in FIG. 26C, a film (thin film) 75 is formed. The film 75 is provided with a through hole 61a′ and a through hole 61b′. The through hole 61a′ and the through hole 61b′ communicate with the through hole 61a and the through hole 61b of the packing 60, respectively. The through hole 61a′ and the through hole 61b′ constitute the flow passage. Similarly, the through hole 62a′ and the through hole 61b′ constitute the flow passage. The film 75 defines the upper surface in the first chamber 11 and the lower surface in the second chamber 12.

The film 75 is provide with a through hole 76 corresponding to the through hole 4 of the partition 3, a through hole 77 to extract extraction electrode 21a, and a through hole 78 to extract extraction electrode 91a.

Subsequently, as shown in FIG. 26D, the upper guide electrode 92 and the extraction electrode 92a thereof are formed on the film 75. A adhesive member (for example, adhesive layer or adhesive agent) 79, and then the partition 3 is placed on the adhesive member 79, thereby fixing the partition 3 on the film 75. At this time, the partition 3 is fixed on the film 75 such that the through hole 4 of the partition 3 communicates with the through hole 76 of the film 7. The through hole 76 is generally larger than the through hole 4.

Subsequently, as shown in FIG. 26A, the packing 60 in FIG. 26B is fixed on the first substrate 50 in FIG. 26A.

Thereafter, the film 75 in FIG. 26D is fixed on the packing 60. The cross-sectional view of the structure in this stage is shown in FIG. 27. The cross-sectional view in FIG. 27 corresponds to a cross-section along the 21-21 line in FIG. 26D.

Subsequently, a structure including the second chamber 12 and the upper electrode 22 is formed in accordance with a well-known method. After that, the direct current power supply 31 and the measurement circuit 32 are connected to the upper electrode 22 in series, and the direct current power supply 93 is connected to the upper guide electrode 92.

Note that, the order of processes shown in FIGS. 26A to 26D can be appropriately changed. For example, the process of FIG. 26B may be before the process of FIG. 26A, or after the process of FIG. 26D.

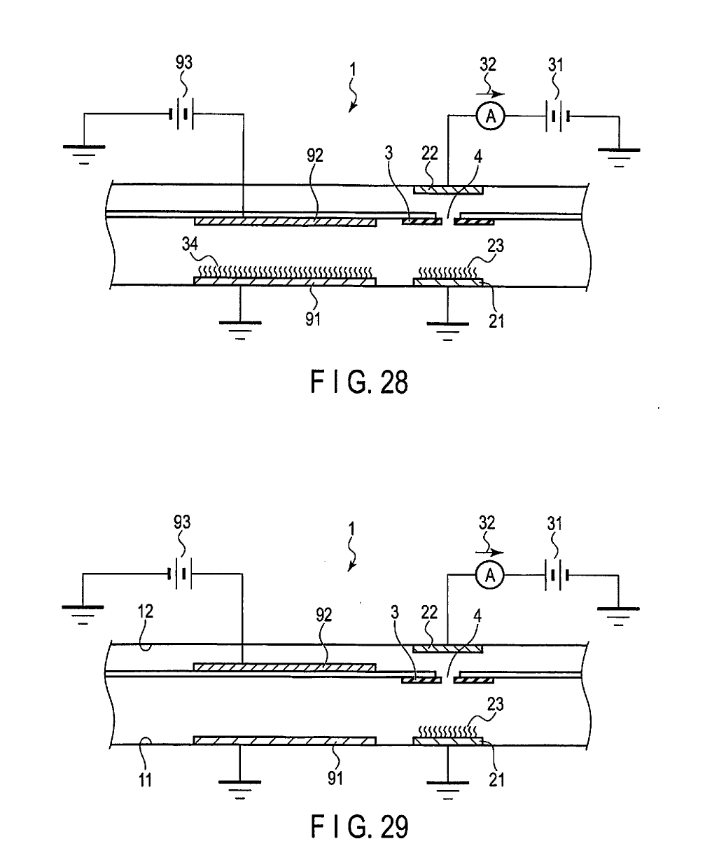

FIG. 28 is a view schematically depicting a detection device 1 of a first variation of the present embodiment. In this variation, a probe 34 for suppressing the detection target from being adsorbed specifically to the lower guide electrode 91.

The probe 34 includes, for example, thiol molecule whose one end is connected to the lower guide electrode 91 and other end is an OH group. That is, the probe 34 includes, for example, a structure in which the second part 23-2 of FIG. 3 is replaced with the OH group. The OH group is exposed on the surface of the lower guide electrode 91. Since the OH group is hydrophilic, the OH group is effective in suppressing the adsorption of the detection target 5.

Note that, both of the lower guide electrode 91 and the upper guide electrode 92 may be provided with the probes 34. The probe 34 (functional group) that is provided to the lower guide electrode 91 may be same as or different from the probe 34 (functional group) that is provided to the upper guide electrode 92. In addition, the probe 34 may be provided to the upper guide electrode 92 alone.

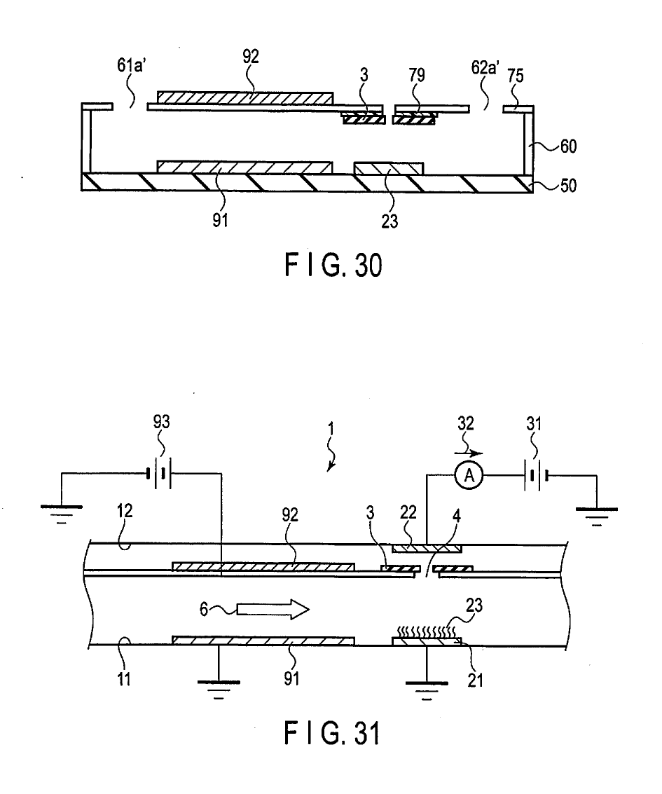

FIG. 29 is a view schematically depicting a detection device 1 according to a second variation of the present embodiment. In the second variation, the upper guide electrode 92 is provided in the second chamber 12. More specifically, the upper guide electrode 92 is provided on lower surface (bottom) in the second chamber 12. FIG. 30 is a cross-sectional view illustrating a structure in a process of manufacturing the detection device 1 according to the second variation, which corresponds to cross-sectional view of FIG. 27. In the second variation, the upper guide electrode 92 is formed on a first plane (surface) of the film 75, and the adhesion member 79 and the partition 3 are formed on the opposite plane (rear surface) side of the first plane of the film 75.

The lower surface in the second chamber 12 (upper surface of the first chamber) is defined by the film 75 shown in FIG. 26C. Since the film 75 is thin, a differential potential can be applied between the lower guide electrode 91 and the upper guide electrode 92 by the direct current power source 93. As a result, the electrical flux lines mentioned above are generated, and the detection target can be guided near the probe 23.

In addition, since the detection liquid is not flown in the second chamber 12, the probe 34 is not required to be provided on the upper guide electrode 92 in the second chamber 12.

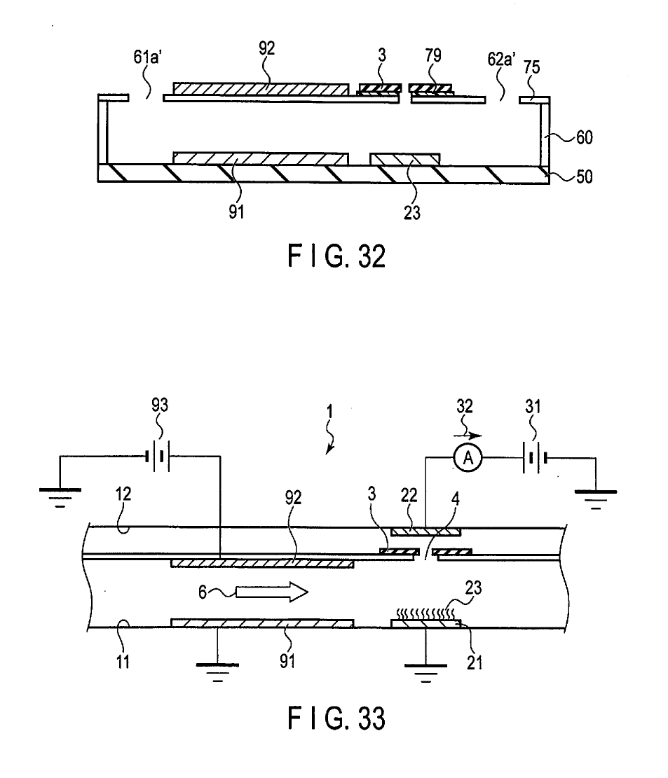

FIG. 31 is a view schematically depicting a detection device 1 according to a third variation of the present embodiment. In the third variation, the partition 3 and the upper guide electrode 92 are provided in the second chamber 12. More specifically, the partition 3 and the upper guide electrode 92 are provided on the lower surface (bottom) in the second chamber 12. FIG. 32 is a cross-sectional view illustrating a structure in a process of manufacturing the detection device 1 according to the third variation, and corresponds to cross-sectional view of FIG. 27. In the third variation, the upper guide electrode 92, the adhesive member 79, and the partition 3 is formed on the surface side of the film 75.

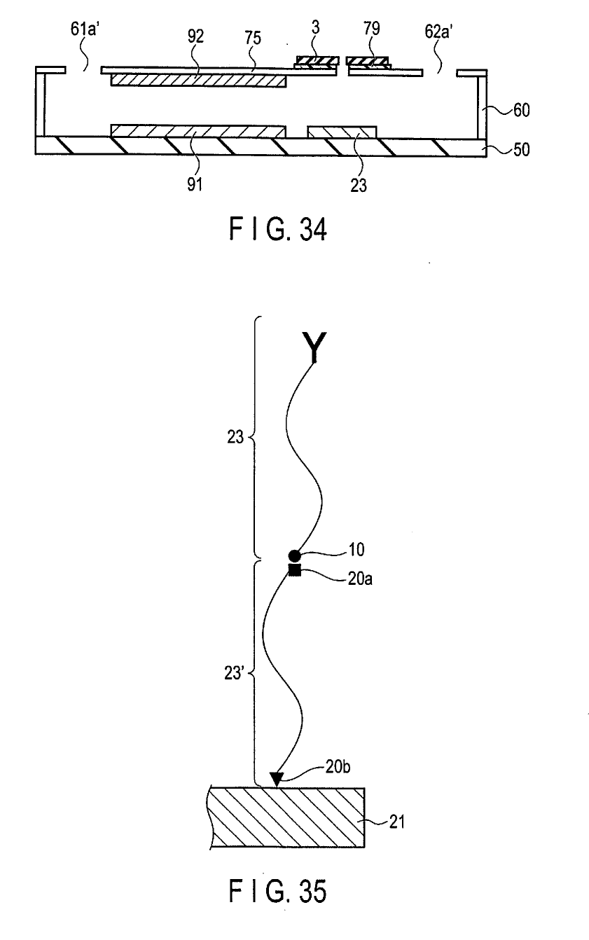

FIG. 33 is a view schematically depicting a detection device 1 according to a fourth variation of the present embodiment. In the fourth variation, the upper guide electrode 92 is provided in the first chamber 11, and the partition 3 is provided in the second chamber 12. More specifically, the upper guide electrode 92 is provided on the upper surface in the first chamber 11, and the partition 3 is provided on the lower surface in the second chamber 12. FIG. 34 is a cross-sectional view illustrating a structure in a process of manufacturing the detection device 1 according to the fourth variation, and corresponds the cross-sectional view of FIG. 27. In the fourth variation, the adhesive member 79 and the partition 3 are formed on the surface side of the film 75, and the upper guide electrode 92 is formed on the rear surface side of the film 75.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A detection device comprising:
a first region configured to be supplied with a first liquid possibly containing a detection target;
a first electrode provided to the first region;
a second region configured to be supplied with a second liquid;
a second electrode provided to the second region;
a partition configured to partition the first region and the second region each other, and comprising a through hole configured to communicate the first region and the second region each other;
a probe detachably connected to the first electrode, and configured to bind specifically to the detection target;
a detacher configured to detach the probe from the first electrode;
a determination unit configured to determine whether the first liquid contains the detection target based on a change of electrical condition between the first electrode and the second electrode in a state where the first region and the second region are supplied with the first liquid and the second liquid, respectively.

2. The detection device of claim 1, wherein the probe comprises a first part detachably connected to the first electrode, and a second part configured to bind specifically to the detection target.

3. The detection device of claim 2, wherein the first part of the probe comprises thiol or disulfide.

4. The detection device of claim 2, wherein the second part of the probe comprises an antibody, nucleic acid, peptide, or chain of sugar.

5. The detection device of claim 1, wherein the first electrode comprises gold, silver, copper, or platinum.

6. The detection device of claim 1, wherein the probe comprises a first probe configured to bind specifically to the detection target, and a second probe configured to bind specifically to another detection target which is different kind from the detection target.

7. The detection device of claim 6, wherein the first probe and the second probe are detachably connected to different regions of the first electrode, respectively.

8. The detection device of claim 6, further comprising a third electrode provided to the first region,
wherein the first probe is detachably connected to the first electrode, and the second probe is detachably connected to the third electrode.

9. The detection device of claim 8, further comprising a fourth electrode provided to the second region and disposed to face the third electrode,
wherein the first electrode is disposed to face the second electrode, and
the partition is provided with a through hole provided between the first electrode and the second electrode and a through hole provided between the third electrode and the fourth electrode.

10. The detection device of claim 1, wherein the detacher comprises a voltage source configured to apply a voltage between the first electrode and the second electrode.

11. The detection device of claim 1, further comprising a guide mechanism configured to guide the detection target possibly contained in the first liquid to the probe.

12. The detection device of claim 11, wherein the guide mechanism comprises:
a fifth electrode provided to the first region,
a sixth electrode facing the fifth electrode, and
a voltage controller configured to control a voltage between the fifth electrode and the sixth electrode.

13. The detection device of claim 1, wherein the second liquid to be supplied to the second region does not contain the detection target.

14. The detection device of claim 1, wherein the detacher further comprises a seventh electrode provided to the first region, and a voltage source connected to the seventh electrode.

15. A detection method using a detection device,
the detection device comprising:
a first region;
a first electrode provided to the first region;
a second region;
a second electrode provided to the second region;
a partition configured to partition the first region and the second region each other, and provided with a through hole configured to communicate the first region and the second region each other; and
a probe detachably connected to the first electrode and configured to bind specifically to a detection target,
the detection method comprising:
supplying a first liquid and a second liquid to the first region and the second region, respectively, the first liquid possibly containing the detection target;
separating the probe from the first electrode;
determining whether the first liquid contains the detection target based on a change of electrical condition between the first electrode and the second electrode.

16. The detection method of claim 15, further comprising guiding the detection target in the first liquid to the probe before separating the probe from the first electrode.

17. The detection method of claim 15, wherein the first liquid is conductive liquid, the second liquid is conductive liquid and does not contain the detection target.

18. An electrode with a probe comprising:
an electrode; and
a probe detachably connected to the electrode and configured to bind specifically to a detection target in a liquid.

19. The electrode of claim 18, wherein the probe is configured to be detached from the electrode by a predetermined treatment.

20. The electrode of claim 19, wherein the probe comprises a first part detachably connected to the electrode, and a second part configured to bind specifically to the detection target, and
the predetermined treatment comprises reducing the first part.
